(19) United States
(12) Patent Application Publication
(10) Pub. No.: US 2014/0356452 A1
(43) Pub. Date: Dec. 4, 2014

(54) Therapeutic Uses of Tetrachlorodecaoxygen (TCDO)

(71) Applicant: IBT USA INC., Greer, SC (US)

(72) Inventors: Otto M. Ah Ching, Greer, SC (US); David Lee Shallcross, Greenville, SC (US); Mark Stephen Kindy, Mount Pleasant, SC (US); Azwan Abdullah Al-Hadi, Bandar Baru Bangi, Selangor (MY)

(21) Appl. No.: 14/373,340

(22) PCT Filed: Jan. 18, 2013
(86) PCT No.: PCT/US13/22236

§ 371 Date: Jul. 18, 2014

Related U.S. Application Data
(60) Provisional application No. 61/588,360, filed on Jan. 19, 2012.

(51) Int. Cl. A61K 33/00

(52) U.S. Cl. CPC A61K 33/00
USPC 424/600

(57) ABSTRACT

The present invention is based in part on the regulation of macrophages. Additionally, the present invention provides methods for the treatment of various diseases and disorders using a compound which regulates macrophage function.

### BACKGROUND OF THE INVENTION

1. Field of the Invention

The present invention relates generally to the field of immunology and more specifically to a method of regulating macrophage function.

2. Background Information

Chronic inflammatory diseases are hypothesized to arise through over-representation of the inflammatory component of the immune cycle, driven in part by trauma or immunologic (e.g., SCI) stimuli which activates NF-κB within macrophages, microglia, endothelial cells and neurons and induction of pro-inflammatory cytokines and chemokines, increase of TNF-α, IL-1b and MCP-1, increase in TNFα and or inappropriately recognized self antigens (autoimmune disease). Continued activation within the macrophage compartment leads to chronic T-cell activation, which acts as a positive feedback mechanism for continued macrophage activation.

Development of a macrophage-targeted therapeutic requires an understanding of the role of macrophages in human disease. The balanced macrophage activation hypothesis of human disease (1) proposes that the major functions of macrophages (phagocytosis, antigen presentation, and inflammation) are components of a host-regulated cyclic process that maintains the homeostasis of macrophage inflammatory activity. The model suggests that chronic diseases arise through over-representation of the inflammatory compartment of the cycle, driven in part by viral (e.g., HIV, HCV), physical trauma to organs and tissue (e.g. SCI) or inappropriately recognized self antigens (i.e., autoimmune disease).

Tetrachlorodecaoxygen (TCDO), which contains chlorite as the active principle, causes profound changes acting on heme complexes found on macrophages. TCDO induces function and activation of gene expression and appears to downregulate inappropriate immunological activation, which leads to a chronic inflammatory response state. The loss of T-cell function observed in many disease states likely requires the involvement of chronically activated macrophages. Therefore, the persistently activated macrophage represents a therapeutic target that is, not highly mutable.

The pharmacological activity of TCDO stems from its ability to downregulate inappropriate immunological activation. TCDO qualitatively and quantitatively modulates the immune response by influencing both cell proliferations and function. The pharmacological effects of TCDO were quantitated in various animal models by measuring the proliferation of immunocompetent cells, phagocytosis index, oxidative burst, cell signaling, and immunoglobulin production. TCDO stimulates phagocytosis, humoral immune response, and cellular defense systems by modifying the function of the monocyte/macrophage system, natural killer (NK) cells, and cytotoxic T-lymphocytes (CTL). Nonclinical and clinical studies have provided evidence that TCDO down-regulates inappropriate immunologic activation through removal of the inflammatory macrophage influence on chronic T cell activation to potentially re-establish immunologic balance. Many chronic diseases are thought to arise through inappropriate immune activation, driven in part by viral (e.g., HIV, HCV) or inappropriately recognized self-antigens (i.e., autoimmune disease). Recent studies have demonstrated the ability of TCDO to specifically regulate macrophage gene expression and thereby inhibit T cell activation. This pharmacologic activity of TCDO may be useful in treating diseases involving; disruption of balanced macrophage activation, such as chronic viral infections, autoimmune disease, allergic hypersensitivity reactions, immune deficiency-associated bacterial and fungal infections, chronic wounds, and a variety of cancers that are outgrowths of chronic inflammation (e.g., lymphoma, pancreatic cancer).

Conventional therapeutic approaches to these chronic inflammatory diseases have employed antiviral/antibacterial, steroidal, combination/cocktail, which have proved to be short-lived, ineffective, and detrimental to patient outcome. New innovative strategies must be directed at understanding the broad interactions/activations of inflammatory mediators, cytokine pathways, and new roles of newly discovered pathways; exclusively addressing virus/bacterial replication, with the goal of decreasing viral load, bacterial resistance. sustainable anti-inflammation, pain cessation improving arsenal of current therapies with robust science.

### SUMMARY OF THE INVENTION

The present invention is based in part on modulating cellular immunity, in particular macrophage function. TCDO regulates macrophage function and may be useful in the treatment of diseases/disorders in which such regulation is therapeutically beneficial.

Accordingly, in one embodiment, the present invention provides a method of reducing and/or inhibiting proinflammatory mediator response in a subject comprising administering to that subject a therapeutically effective amount of Tetrachlorodecaoxygen (TCDO). One embodiment of the present invention provides a method of modifying cellular immunity in a subject comprising administering to that subject a therapeutically effective amount of TCDO. In a further embodiment, the present invention provides a method of controlling chronic inflammation in a subject comprising administering to that subject a therapeutically effective amount of TCDO. In one embodiment, the present invention provides a method of regulating macrophage function in a subject comprising administering to that subject a therapeutically effective amount of TCDO.

In one aspect, the subject has a macrophage mediated disease. In an additional aspect, the macrophage mediated disease is spinal cord injury, traumatic brain injury, chronic trauma encephalitis, viral/bacterial encephalitis/meningitis, schizophrenia, fibromyalgia, chronic fatigue syndrome, chronic depression, spasticity, Lyme's disease, multiple sclerosis, myocardial infarction, stroke, viral infection, microbial infection, MRSA, malaria, dengue fever, filiriasis, Leschmaniasis, diabetes mellitus type II, dementia, Alzheimer's disease or Parkinson's disease. In a further aspect, the subject is in need of wound healing/repair, burns, neuropathy, or pain cessation. In yet a further aspect, the subject has diabetes mellitus type II.

In another embodiment, the invention provides a method of treating a subject to produce pain cessation comprising administering to the subject a therapeutically effective amount of TCDO. In a further embodiment, the present invention provides, a method of treating a subject to produce an analgesic effect comprising administering to the subject a therapeutically effective amount of TCDO. In an additional embodiment, the invention provides a method of promoting wound healing in a subject comprising administering to the subject a therapeutically effective amount of TCDO.

In one aspect TCDO is administered by parenteral, topical, intravenous, oral, subcutaneous, intraarterial, intracranial, intraperitoneal, intranasal or intramuscular means.

In an additional aspect, TCDO is administered at a dose from about 0.1 mL/kg body weight to 1.5 mL/kg body weight. In a preferred aspect, TCDO is administered at a dose of about 0.5 mL/kg body weight to 0.75 mL/kg body weight. In another preferred aspect, TCDO is administered at a dose of about 0.5 mL/kg body weight.

In one aspect, the subject receives at least one cycle of TCDO, wherein a cycle consists of the patient is administered TCDO for about five consecutive days and then does is not administered TCDO for at least about 16 consecutive days. In a further aspect, the subject is administered 1, 2, 3, 4, 5, or 6 cycles of TCDO.

### BRIEF DESCRIPTION OF THE DRAWINGS

FIG. 1 is a depiction of the balanced macrophage activation hypothesis of human disease.

FIG. 2 is a depiction demonstrating the effect of a TCDO therapeutic agent on chronic inflammation.

FIG. 3 is a chart demonstrating the effects of TCDO on lesion volumes in the rat following spinal cord injury. All rats were subjected to spinal cord injury followed by 6 weeks of recovery. Animals were injected with Saline, NTS-Saline or TCDO solutions. Animals were sacrificed on day 42 and processed to determine the lesion volume. Treatment with TCDO reduced the lesion volume by 25, 50 and 65% but the change were statistically significant relative to the Saline group (control group) only in the 5×10⁴ and 10×10⁴ dose groups. * and ** Significant decrease in lesion volume * P=0.0004 **P<0.0001.

FIG. 4 is a chart demonstrating the open-field (BBB) behavioral measurements in rats subjected to spinal cord injury. All rats were either sham (not-injured) control or subjected to spinal cord injury and were injected with Saline or received TCDO solutions. Behavioral analysis was measured once a week for a period of 6 weeks post-injury. Only raw data are presented in the figure. *p<0.05 against saline and control groups.

FIG. 5 is a chart showing changes in inflammatory markers. A. GFAP immunoreactivity following TCDO administration. B. IL-1 immunoreactivity following WF-10 administration. C. CD-68 Immunoreactivity following WF-10 administration. All rats were either sham (not-injured) control or subjected to spinal cord injury and were injected with Saline or received TCDO (WF-10) solutions. Immunohistochemical analysis was measured at the end of the study (N=10 with 3 sections used for analysis per animal). Only raw data are presented in the figure.

FIG. 6 is a depiction of the brains of APP transgenic mice (3 months of age) in the absence (A) and presence of TCDO (WF-10, B). WF-10 was injected at 1/400 dilution intravenously on a daily basis for 90 days starting prior to amyloid deposition.

FIG. 7 depicts the method of action of TCDO to aid in wound healing.

### DETAILED DESCRIPTION OF THE INVENTION

The present invention is based in part modulating cellular immunity, in particular macrophage function. TCDO regulates macrophage function and may be useful in the treatment of diseases/disorders in which such regulation is therapeutically beneficial.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “macrophage mediated disease” refers to conditions which result from abnormal activity of the body's immune system involving macrophages. Macrophage mediated diseases include, but are not limited to, spinal cord injury, traumatic brain injury, multiple sclerosis, myocardial infarction, stroke, viral infection, microbial infection, diabetes mellitus type II, wound healing, pain/chronic pain, Alzheimer's disease and Parkinson's disease.

As used herein, the term “TCDO” refers to the compound Tetrachlorodecaoxygen. The terms “WF10”, “WF10/MACROSTAT”, “MACROSTAT”, “OXO-K933” and “Nuderm” refer to pharmaceutical formulations of TCDO.

The term “therapeutically effective amount” refers to an amount of therapeutic agent effective to produce a therapeutically beneficial response in a subject or mammal.

In one embodiment, the present invention provides a method of reducing and/or inhibiting proinflammatory mediator response in a subject comprising administering to that subject a therapeutically effective amount of Tetrachlorodecaoxygen (TCDO). One embodiment of the present invention provides a method of modifying cellular immunity in a subject comprising administering to that subject a therapeutically effective amount of TCDO. In a further embodiment, the present invention provides a method of controlling chronic inflammation in a subject comprising administering to that subject a therapeutically effective amount of TCDO. In one embodiment, the present invention provides a method of regulating macrophage function in a subject comprising administering to that subject a therapeutically effective amount of TCDO.

In one aspect, the subject has a macrophage mediated disease. In an additional aspect, the macrophage mediated disease is spinal cord injury, traumatic brain injury, multiple sclerosis, myocardial infarction, stroke, viral infection, microbial infection, diabetes mellitus type II, Alzheimer's disease or Parkinson's disease. In a further aspect, the subject is in need of wound healing or pain cessation. In yet a further aspect, the subject has diabetes mellitus type II.

TCDO is a macrophage regulator that appears to act through removal of the inflammatory macrophage influence on chronic T cell activation to potentially restore immunologic balance.

Macrophages are cells produced by the differentiation of monocytes in tissues. Monocytes and macrophages are phagocytes. Macrophages function in both non-specific defense (innate immunity) as well as help initiate specific defense mechanisms (adaptive immunity) of vertebrate animals. Their role is to phagocytose cellular debris and pathogens, either as stationary or as mobile cells. They also stimulate lymphocytes and other immune cells to respond to pathogens. They are specialized phagocytic cells that attack foreign substances, infectious microbes and cancer cells through destruction and ingestion. Macrophages can be identified by specific expression of a number of proteins including CD14, CD40, CD11b, CD64, F4/80 (mice)/EMR1 (human), lysozyme M, MAC-1/MAC-3 and CD68 by flow cytometry or immunohistochemical staining.

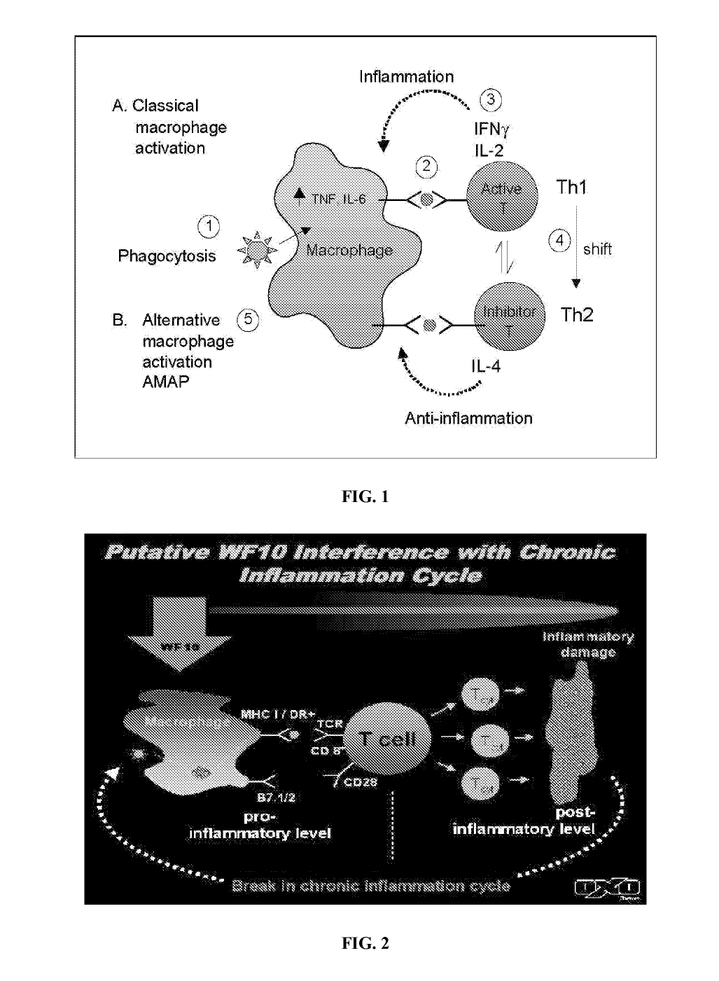

Development of a macrophage-targeted therapeutic requires an understanding of the role of macrophages in human disease. The balanced macrophage activation hypothesis of human disease proposes that the major functions of macrophages (phagocytosis, antigen presentation, and inflammation) are components of a host-regulated cyclic process that maintains the homeostasis of macrophage inflammatory activity. The model suggests that chronic diseases arise through over-representation of the inflammatory compartment of the cycle, driven in part by viral (e.g., HIV, HCV), physical trauma to organs and tissue (e.g. SCI) or inappropriately recognized self antigens (i.e., autoimmune disease). This paradigm is proposed in FIG. 1.

Phagocytosis. The first step in macrophage activation is phagocytosis. Macrophages engulf pathogens, such as, bacteria, fungi and viruses. Upon successful phagocytosis of a foreign substance, the macrophage processes this material through a proteolytic pathway, cutting individual proteins into small peptides that are then involved in the second step of macrophage activation: the presentation of antigens to T-cells. Phagocytosis is a basic macrophage function, which explains the derivation of the name ‘macrophage from ‘macro’, meaning big, and ‘phage’, meaning eater, thereby conferring on the macrophage the term ‘big-eater”.

Antigen presentation. After foreign materials have been cut into peptides, macrophages present antigen to T-lymphocytes utilizing the major histocompatibility antigens class 1 (HLA) and class 2 (DR) and initiate expansion of a normal immune response (step 2). T-cell activation predominantly occurs through this antigen-presenting-cell function. Standard cytotoxic T-cells specific for virus-infected cells, cancers or fungi are developed, which ultimately leads to their successful immunological clearance. Upon successful activation, T-cells express various activation antigens, such as CD38, and secrete factors, such as IL-2, which allows T-cells to proliferate, and IFNγ, which causes further macrophage activation and step 3.

Classical macrophage activation. A product of T-cell activation, IFNγ Induces full Inflammatory changes and classical macrophage activation. This activation causes upregulation of inflammatory cytokines, such as IL-1, IL-6, and TNFα. The macrophage in this state is extremely inflammatory and causes secondary effects, such as fevers, and when chronically stimulated, weight loss and further non-specific activation of immunological responses.

Th1 to Th2 (active-to-inhibitory T-cell) shift. During the initiation of a cellular response, which ultimately leads to production of cytotoxic T-cells and IL-2-producing Th1 cells, a second major class of T-cells, the Th2 cell, is induced. Th2 cells are involved in B-cell activation and proliferation, hypergammaglobulinemia, upregulation of IgE, eosinophilia and allergic reactions. As a net result, excess IL-10 production shuts off step 2. The Th1 and Th2 cell activation process occurs virtually simultaneously in vitro (and likely in vivo), although classical immunological responsiveness, as measured by T-cell proliferation, predominantly considers the Th1-like response. A key feature of the Th2 response is production of IL-4, which is known to activate the alternative macrophage activation pathway (AMAP).

TCDO appears to downregulate inappropriate immunologic activation. Previous studies have shown that TCDO reduces antigen presentation in vitro, and recent gene expression studies, provide evidence that TCDO reduces inappropriate T cell activation by regulating macrophage function. Inhibition of proinflammatory mediators by proper macrophage regulation induced by TCDO via antigen presentation leads to anergic reaction (tolerance). Agents that directly influence macrophage activation would be predicted by this model to cause secondary effects on T-cell activation.

Chronic inflammatory diseases are hypothesized to arise through over-representation of the inflammatory component of the immune cycle, driven in part by trauma or immunologic (e.g., SCI) stimuli which activates NF-kB within macrophages, microglia, endothelial cells and neurons and induction of pro-inflammatory cytokines and chemokines, increase of TNF-α, IL-1b and MCP-1, increase in TNFα and or inappropriately recognized self antigens (autoimmune disease). According to the model, continued activation within the macrophage compartment leads to chronic T-cell activation, which acts as a positive feedback mechanism for continued macrophage activation. This pharmacologic activity of TCDO may be useful in treating diseases involving disruption of balanced macrophage activation, such as chronic viral infections, autoimmune disease, allergic hypersensitivity reactions, immune deficiency-associated bacterial and fungal infections, chronic wounds, and a variety of cancers that are outgrowths of chronic inflammation (e.g., lymphoma), multiple sclerosis, SCI, traumatic brain injury, Parkinson's disease, Alzheimer's disease and arterial-venous vascular disease.

There is considerable evidence for the immune-modulating action of TCDO, particularly to induce natural defense mechanism and to inhibit inappropriately activated adaptive immune response. TCDO has been shown to stimulate phagocytosis, humoral immune response, and cellular defense systems by modifying the function of the monocyte/macrophage system, natural killer (NK) cells, and cytotoxic T-lymphocytes (CTL). TCDO increased macrophage phagocytosis, stimulated an oxidative burst in monocytes, decreased macrophage antigen presentation, and inhibited TNF-α expression in macrophages. A proposed concept of ‘balanced macrophage activation’ offers an explanation of the role of immune dysfunction and excessive activation in the pathogenesis of diverse diseases such as chronic viral infections, autoimmune disease, allergic hypersensitivity reactions, immune deficiency-associated bacterial and fungal infections, chronic wounds, and a variety of cancers that are outgrowths of chronic inflammation (e.g., lymphoma, pancreas cancer). These pharmacological activities of TCDO suggest it may be of therapeutic benefit by reversing the immune dysfunction and restoring balanced macrophage activation.

In a series of in vitro experiments in monocytes, macrophages and lymphocytes TCDO was observed to increase phagocytosis, stimulate an oxidative burst in monocytes, decrease macrophage tumor necrosis factor (TNF)-α expression, decrease antigen presentation and enhance macrophage-fibroblast cooperation. In vitro, TCDO also displayed anti-HIV properties that did not appear to be involved in the drug's macrophage targeted mechanism of action. In vivo studies investigated the effects of TCDO on monocytes, macrophages and lymphocytes, on humoral and cellular immunity, and on response to local or total body irradiation. TCDO increased the number of macrophages infiltrating a skin blister in a human wound-healing model.

In rats, TCDO increased the proportion of granulocytes, peripheral blood monocytes (PBMCs) and large granular lymphocytes (LGLs), and stimulated erythropoiesis after total body X-irradiation. In mice, TCDO stimulated regeneration of hematopoietic stem cells receiving sublethal doses of J-irradiation. In other studies, TCDO displayed direct antitumor effects against radiation-induced, chemical induced and metastatic malignant and benign tumors. TCDO altered proportions of T-helper and T suppressor/cytotoxic cells in spleen and thymus and increased both the humoral and cellular immune responses measured by the Jerne plaque and footpad swelling tests, respectively. TCDO (0.2 ml/kg iv) administered twice during the day before iv administration of lipopolysaccharide (LPS) from E coli 055B5 reduced the sensitivity of BALB/cABOM mice to the endotoxin, increasing the LD50 value from 346 to 518 mg. TCDO attenuation of endotoxin toxicity is consistent with the downregulation of expression of TNF-α observed in vitro.

In a phagocytosis assay, ingestion of E. coli was maximal at a concentration corresponding to a TCDO dose of 0.5-mL/kg-body weight. Upon administration of TCDO daily for 90 days to rats at doses ranging from 0.33 to 2.0 mL/kg body weight, blood levels of monocytes and large granular lymphocytes increased significantly to greater than three times baseline value within four days. This elevated level was maintained for the remainder of the dosing period. After discontinuation of TCDO administration, the monocyte level gradually declined and returned to normal value after 30 days. Sixteen days after discontinuation of TCDO, the monocyte level was approximately 50% elevated over baseline.

Additionally, TCDO has been shown in clinical trials to be therapeutically beneficial in patients suffering from autoimmune disease with skin manifestations. Patients suffering from chronic actinic dermatitis developed photosensitivity 5-10 years prior to the study. The patients' faces become mask like and deteriorate with sunlight and smoke. The patients' skin becomes discolored and dry with bleeding lesions. Steroid therapy caused blindness requiring lens replacement. Patients were successfully treated with two cycles of TCDO at 0.5 mL/kg body weight. Pemphigus vulgaris is a disease in which autoantibodies are directed against the cell surface of keratinocytes. Treatment with high does steroids and cyclophosphamide controls the blisters but puts the patients at risk for developing sepsis and the blister wounds do not heal. Patients were successfully treated with two cycles of TCDO at 0.5 mL/kg body weight. A patient suffering from pustular psoriasis experienced widespread pustules with accumulated blood vessels underneath with itching and pain for greater than 5 years. Treatment with retinoic drugs was not successful The patient was successfully treated with two cycles of TCDO at 0.5 mL/kg body weight. A patient suffering from HIV associated psoriasis presented with psoriasis affecting the entire body. The condition was previously treated with methotrexate which caused a significant decrease in white blood cells. The patient was successfully treated with four cycles of TCDO at 0.5 mL/kg body weight. A patient suffering from eczema presented with eczema on the hands and feet with very dry skin. Treatment with steroid creams caused deterioration of the skin. The patient was successfully treated with four cycles of TCDO at 0.5 mL/kg body weight.

Further, TCDO has been shown to be effective for treatment for healing wounds when applied topically. In a study with 271 patients, TCDO was shown to be significantly superior to saline for wound cleaning and formation of new tissue when applied topically. Further in a randomized clinical study, TCDO was shown to be superior to PVP-iodine treatment leading to a 74.9% reduction in wound surface. Additionally, TCDO was shown to reverse the effects of cortisone on wounds when applied topically.

In summary, TCDO has pro-oxidative activities, endogenously produced in particular microenvironments of the human body, such as the intestinal mucosa, represent a physiologic principle of down-regulation of unwanted potentially harmful antigen-driven immune response. TCDO represents a compound exhibiting pro-oxidative activity that can safely be administered to humans. TCDO exerts anti-inflammatory activities toward in vitro stimulated lymphocytes and, in a more pronounced fashion, monocytes. TCDO lowers the expression of genes encoding for pro-inflammatory cytokines in vitro and, upon application to humans with up-regulated genes, in vivo.

In one embodiment, the present invention provides a method of treating a subject having a macrophage mediated disease comprising administering to the subject a therapeutically effective amount of TCDO. In one aspect, the macrophage mediated disease is spinal cord injury, traumatic brain injury, multiple sclerosis, myocardial infarction, stroke, viral infection, microbial infection, diabetes mellitus type II, pain/chronic pain, wound healing, Alzheimer's disease and Parkinson's disease. In another embodiment, the invention provides a method of treating a subject to produce pain cessation comprising administering to the subject a therapeutically effective amount of TCDO.

In a further embodiment, the present invention provides, a method of treating a subject to produce an analgesic effect comprising administering to the subject a therapeutically effective amount of TCDO. In an additional embodiment, the invention provides a method of promoting wound healing in a subject comprising administering to the subject a therapeutically effective amount of TCDO.

### Acute and Chronic Neuroinflammation

The word neuroinflammation has come to stand for chronic, inflammation-like glial responses that may produce neurodegenerative symptoms such as plaque formation, dystrophic neurite growth, and excessive tau phosphorylation It is important to distinguish between acute and chronic neuroinflammation. Acute neuroinflammation is generally caused by some neuronal injury after which microglia migrate to the injured site engulfing dead cells and debris. The term neuroinflammation generally refers to more chronic, sustained injury when the responses of microglial cells contribute to and expand the neurodestructive effects, worsening the disease process.

When microglia are activated they take on an amoeboid shape and they increase their gene expression. Increased gene expression leads to the production of numerous potentially neurotoxic mediators. These mediators are important in the normal functions of microglia and their production is usually decreased once their task is complete. In chronic neuroinflammation, microglia remain activated for an extended period during which the production of mediators is sustained longer than usual. This increase in mediators contributes to neuronal death.

Neuroinflammation is unique from inflammation in other organs, but does include some similar mechanisms such as the localized production of chemoattractant molecules to the site of inflammation. The following list contains a few of the numerous substances that are secreted when microglia are activated:

### Cytokines

Microglia activate the proinflammatory cytokines; IL-1α, IL-1β and TNF-α in the CNS Cytokines play a potential role in neurodegeneration when microglia remain in a sustained activated state. Direct injection of the cytokines IL-1α, IL-1β and TNF-α into the CNS result in local inflammatory responses and neuronal degradation. This is in contrast with the potential neurotrophic (inducing growth of neurons) actions of these cytokines during acute neuroinflammation.

### Chemokines

Chemokines are cytokines that stimulate directional migration of inflammatory cells in vitro and in vivo. Chemokines are divided into four main subfamilies: C, CC, CXC, and CX₃C. Microglial cells are sources of some chemokines and express the monocyte chemoattractant protein-1 (MCP-1) chemokine in particular. Other inflammatory cytokines like IL-10 and TNF-α, as well as bacterial-derived Lippopolysaccharide (LPS) may stimulate microglia to produce MCP-1, MIP-1α, and MIP-1β. Microglia can express CCR3, CCR5, CXCR4, and CX3CR1 in vitro. Chemokines are proinflammatory and therefore contribute to the neuroinflammation process.

### Proteases

When microglia are activated they induce the synthesis and secretion of proteolytic enzymes that are potentially involved in many functions. There are a number of proteases that possess the potential to degrade both the extracellular matrix and neuronal cells that are in the neighborhood of the microglia releasing these compounds. These proteases include; cathepsins B, L, and S, the matrix metalloproteinases MMP-1, MMP-2, MMP-3, and MMP-9, and the metalloprotease-disintegrin ADAM8 plasminogen which forms outside microglia and degrades the extracellular matrix. Cathepsin B, MMP-1 and MMP-3 have been found to be increased in Alzheimer's disease (AD) and cathepsin B is increased in multiple sclerosis (MS). Elastase, another protease, could have large negative effects on the extracellular matrix

### Amyloid Precursor Protein

Microglia synthesize amyloid precursor protein (APP) in response to excitotoxic injury. Plaques result from abnormal proteolytic cleavage of membrane bound APP. Amyloid plaques can stimulate microglia to produce neurotoxic compounds such as cytokines, excitotoxin, i NOS-nitric oxide and lipophylic amines, which all cause neural damage. Plaques in Alzheimer's disease contain activated microglia. A study has shown that direct injection of amyloid into brain tissue activates microglia, which reduces the number of neurons. Microglia have also been suggested as a possible source of secreted α amyloid.

### Aging

Microglia undergo a burst of mitotic activity during injury; this proliferation is followed by apoptosis to reduce the cell numbers back to baseline. Activation of microglia places a load on the anabolic and catabolic machinery of the cells causing activated microglia to die sooner than non-activated cells. To compensate for microglial loss over time, microglia undergo mitosis and bone marrow derived progenitor cells migrate into the brain via the meninges and vasculature.

Accumulation of minor neuronal damage that occurs during normal aging can transform microglia into enlarged and activated cells. These chronic, age-associated increases in microglial activation and IL-1 expression may contribute to increased risk of Alzheimer's disease with advancing age through favoring neurotic plaque formation in susceptible patients. DNA damage might contribute to age-associated microglial activation. Another factor might be the accumulation of advanced glycation end products, which accumulate with aging. These proteins are strongly resistant to proteolytic processes and promote protein cross-linking Research has discovered dystrophic (defective development) human microglia. “These cells are characterized by abnormalities in their cytoplasmic structure, such as deramified, atrophic, fragmented or unusually tortuous processes, frequently bearing spheroidal or bulbous swellings.” The incidence of dystrophic microglia increases with aging. Microglial degeneration and death have been reported in research on Prion disease, Schizophrenia and Alzheimer's disease, indicating that microglial deterioration might be involved in neurodegenerative diseases A complication of this theory is the fact that it is difficult to distinguish between “activated” and “dystrophic” microglia in the human brain

### Treatment of Neuroinflammation

### Inhibition of Activation/Anergy-Tolerance

One way to control neuroinflammation is to inhibit microglial activation. Studies on microglia have shown that they are activated by diverse stimuli but they are dependent on activation of mitogen-activated protein kinase (MAPK). Previous approaches to down-regulate activated microglia focused on immunosuppressants. Presently, a promising treatment is TCDO, which down-regulates TNF α-inhibiting compound that also down-regulates IL-1, IL2, Recent study shows that TCDO, Kindy et al. suppresses the production of proinflammatory cytokines and superoxide anion by activated microglia.

### Regulation of Chemokine Receptor

The chemokine receptor, CX3CR1, is expressed by microglia in the central nervous system Fractalkine (CX3CL1) is the exclusive ligand for CX3CR1 and is made as a transmembrane glycoprotein from which a chemokine can be released. Cardona, et al. stated in 2006 that “using three different in vivo models, we show that CX3CR1 deficiency dysregulates microglial responses, resulting in neurotoxicity.” Further studies into how CX3CR1 regulates microglial neurotoxicity could lead to new therapeutic strategies for neuroprotection

### Inhibition of Amyloid Deposition

Inhibitors of amyloid deposition include the enzymes responsible for the production of extracellular amyloid such as β-secretase and γ-secretase inhibitors. Currently the γ-secretase inhibitors are in phase II clinical trials as a treatment for Alzheimer's disease but they have immunosuppressive properties, which could limit their use. Another strategy involves increasing the antibodies against a fragment of amyloid. This treatment is also in phase II clinical trials for the treatment of Alzheimer's disease.

### Inhibition of Cytokine Synthesis

Glucocorticosteroids are anti-inflammatory steroids that inhibit both central and peripheral cytokine synthesis and action. In a recent study, both Lovostatin and sodium phenylacetate were found to inhibit TNF-α, IL-1β, and IL-6 in rat microglia. This shows that the mevalonate pathway plays a role in controlling the expression of cytokines in microglia and may be important in developing drugs to treat neurodegenerative diseases. Naltrexone may pose a solution to the inflammatory mediators produced by microglia. Although naltrexone's main action is to competitively bind to opioid receptors thereby upregulating the number of receptors; mover over, it is used to help patients with an opioid dependency. New research shows that low-dose naltrexone can inhibit cytokine synthesis of microglia cells. This mechanism is still in its infancy, but it has proven to help some patients suffering from fibromyalgia syndrome.

### Role of Microglia in Neurodegeneration

Neurodegenerative disorders are characterized by progressive cell loss in specific neuronal populations.” Many of the normal trophic functions of glia may be lost or overwhelmed when the cells become chronically activated in progressive neurodegenerative disorders, for there is abundant evidence that in such disorders, activated glia play destructive roles by direct and indirect inflammatory attack.” The following are prominent examples of microglial cells' role in neurodegenerative disorders.

### Alzheimer's Disease

In one embodiment, the present invention provides a method of treating a subject having Alzheimer's Disease comprising administering to the subject a therapeutically effective amount of TCDO.

Alzheimer's disease (AD) is a progressive, neurodegenerative disease where the brain develops abnormal clumps (amyloid plaques) and tangled fiber bundles (neurofibrillary tangles).

Alzheimer's disease (AD) is the most common form of dementia. There is no cure for the disease, which worsens as it progresses, and eventually leads to death. The cause and progression of Alzheimer's disease are not well understood. Research indicates that the disease is associated with plaques and tangles in the brain. Current treatments only help with the symptoms of the disease. There are no available treatments that stop or reverse the progression of the disease. http://en.wikipedia.org/wiki/Non-steroidal_anti-inflammatory_drug”

Various inflammatory processes and cytokines may also have a role in the pathology of Alzheimer's disease. Inflammation is a general marker of tissue damage in any disease, and may be either secondary to tissue damage in AD or a marker of an immunological response

The CNS contains two distinct populations of mononuclear phagocytes: the microglial cells of the brain parenchyma and the macrophages that reside in perivascular spaces, meningeal folds, and the choroid plexus. These two classes of phagocytes are known to differ in the production of specific cytokines, cell surface immune antigen expression, and ability to promote innate versus adaptive immune responses. There are several characteristics of peripheral macrophages that make them efficient professional Aβ phagocytes. First, monocyte precursors that develop into peripheral macrophages have a dynamic life cycle, while brain-resident microglia have a prolonged lifespan and limited capacity to divide. Further, it has been suggested that peripheral macrophages can enter and exit the CNS compartment throughout life. In contrast, microglia are permanent CNS residents, and their phenotype is significantly influenced by habitation there. Brain-resident microglia are therefore more tightly regulated spatially and temporally, allowing for precise CNS-tuned immune responses but restricted functional repertoire. In their quiescent (resting) state, human brain-resident microglia in general lack MHC-II (HLA-DR) antigen, cytokine expression, CD45 antigen, and other surface immune molecules required for antigen presentation and phagocytosis. Alternatively, peripheral mononuclear phagocytes constitutively express HLA-DR and are capable of the full repertoire of innate immune responses. Thus, while microglia serve as sentinels to disruption of homeostasis in neural tissue and have limited innate immune responses, peripheral mononuclear phagocytes are unrestricted in their abilities to engulf and digest cellular debris and pathogens.

Given that peripheral macrophages originate from monocyte/dendritic progenitors present in the bone marrow and the circulation, these cells can be therapeutically targeted in a minimally invasive fashion in the periphery. Conversely, targeting brain-resident microglia for therapeutics can be challenging due to the presence of the BBB. Moreover, given the fact that peripheral macrophages are generally regarded as professional phagocytes, they have greater Aβ phagocytic potential than their immune-repressed microglia cousins.

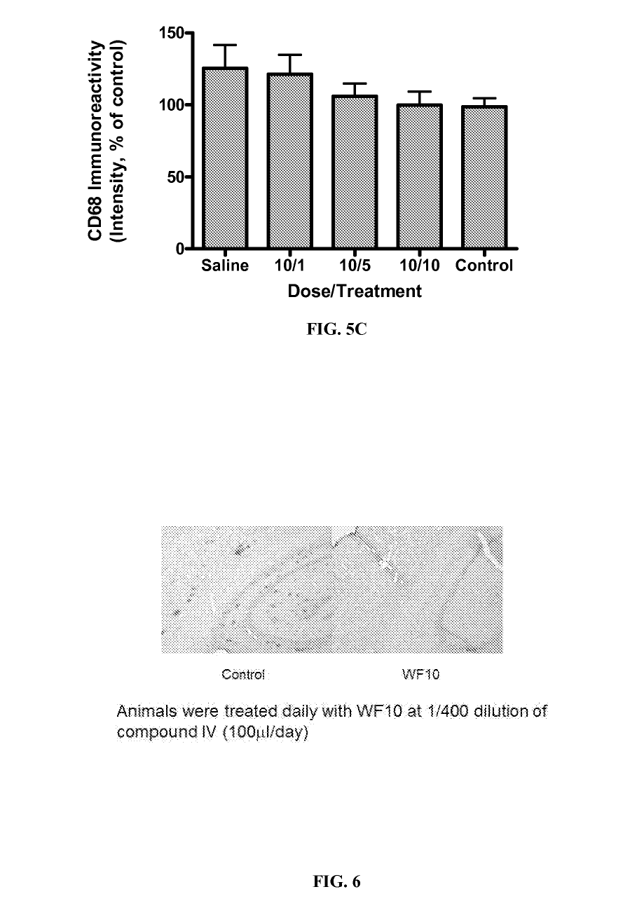

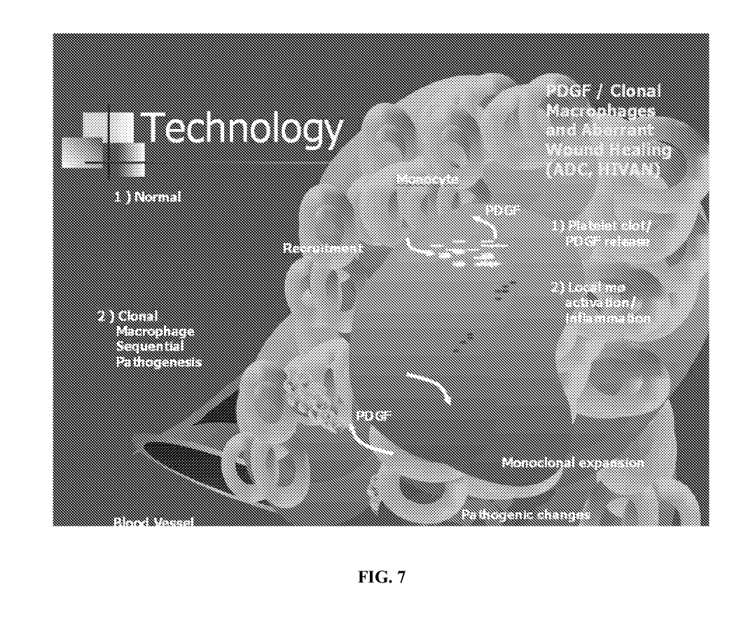

Research has indicated that macrophages may be beneficial in the treatment of Alzheimer's disease. As such, treatment with an immunologic agent which regulates macrophages, such as TCDO, may be therapeutically useful in the treatment of Alzheimer's disease. Results of using TCDO in an Alzheimer's animal model are shown in FIG. 6.

### Parkinson's Disease

In one embodiment, the present invention provides a method of treating a subject having Parkinson's disease comprising administering to the subject a therapeutically effective amount of TCDO.

Parkinson's is a movement disorder in which the dopamine-producing neurons in the brain do not function as they should.

Parkinson's disease (PD) is a degenerative disorder of the central nervous system. The motor symptoms of Parkinson's disease result from the death of dopamine-generating cells in the substantia nigra, a region of the midbrain; the cause of this cell death is unknown. Early in the course of the disease, the most obvious symptoms are movement-related; these include shaking, rigidity, slowness of movement and difficulty with walking and gait. Later, cognitive and behavioral problems may arise, with dementia commonly occurring in the advanced stages of the disease.

Modern treatments are effective at managing the early motor symptoms of the disease, mainly through the use of levodopa and dopamine agonists. As the disease progresses and dopaminergic neurons continue to be lost, these drugs eventually become ineffective at treating the symptoms and at the same time produce a complication called dyskinesia, marked by involuntary writhing movements. Research directions include investigations into new animal models of the disease and of the potential usefulness of gene therapy, stem cell transplants and neuroprotective agents. Recently, it was found that genes involved in the immune response is linked to the risk of developing Parkinson's disease, strengthening a theory that PD may result partly from harmful immune reactions such as inflammation, infections or autoimmunity.

Another prominent pathological feature of PD brains is the presence of a robust inflammatory response mediated by activated microglia and reactive astrocytes in affected areas of the substantia nigra (SN). As a result of pathogen invasion or tissue damage, microglia promote an inflammatory response that serves to further engage the immune system by recruiting other cells to the site of brain lesion, and initiate tissue repair. However, uncontrolled inflammation may result in production of neurotoxic factors that can be highly detrimental. Inflammation in the central nervous system (CNS) and sustained overactivation of microglia, i.e. reactive microgliosis, are currently believed to be actively involved in the pathogenesis of various neurodegenerative diseases including PD, Alzheimer's disease, multiple sclerosis (MS), and amyotrophic lateral sclerosis (ALS).

As such treatment using an agent which regulates macrophages, such as TCDO, may be therapeutically beneficial in the treatment of PD.

### Traumatic Brain Injury

In one embodiment, the present invention provides a method of treating a subject having traumatic brain injury comprising administering to the subject a therapeutically effective amount of TCDO.

Traumatic brain injury (TBI), also known as intracranial injury, occurs when an external force traumatically injures the brain. TBI can be classified based on severity, mechanism (closed or penetrating head injury), or other features (e.g., occurring in a specific location or over a widespread area).

Acute TBI is characterized by two injury phases, primary and secondary. The primary brain injury is the direct injury to the brain cells incurred at the time of the initial impact. This results in a series of, biochemical processes which then result in secondary brain injury. There is a complex cascade of cellular inflammatory response following TBI which propagates secondary brain damage. This inflammatory process lasts from hours to days contributing continuously to secondary brain damage. The inflammatory response resulting from an acute TBI is not limited to the brain and multiple organ dysfunction syndromes are commonly seen. The major molecules in the brain involved in this cascade are growth factors, catecholamines, neurokinins, cytokines and chemokines.

Secondary injury begins minutes after injury and can continue years after the initial insult. Mechanisms implicated in secondary injury after TBI include glutamate excitotoxicity, blood-brain barrier disruption, secondary hemorrhage, ischemia, mitochondrial dysfunction, apoptotic and necrotic cell death, and inflammation. As the primary mediators of the brain's innate immune response to infection, injury, and disease, microglia react to injury within minutes. In fact, microglia may represent the first line of defense following injury.

Microglia also produce a number of neuroprotective substances after injury, including anti-inflammatory cytokines (IL-10, IL-1 receptor antagonist (II-1ra)) and neurotrophic factors (nerve growth factor, transforming growth factor β (TGF-β). IL-10, which is elevated acutely after injury in humans, has been shown to have beneficial effects in experimental models of injury). These neuroprotective effects may be a result of suppressed microglial production of proinflammatory cytokines TGF-β also has also been shown to have neuroprotective effects after injury, including improved function, decreased lesion size, and decreased iNOS production.

The rise in inflammatory cytokines (e.g. IL-6) following TBI is a double edged sword; both neurotoxicity and neuroprotection may be induced by it. Inflammatory cytokines facilitate neurotoxicity by encouraging excitotoxicity and the inflammatory response, but simultaneously they facilitate the neurotrophic mechanisms and induction of cell growth factors which are neuroprotective.

As such treatment using an agent which regulates macrophages, such as TCDO, may be therapeutically beneficial in the treatment of TBI.

### Spinal Cord Injury

In one embodiment, the present invention provides a method of treating a subject having a spinal cord injury comprising administering to the subject a therapeutically effective amount of TCDO.

There are approximately 14,000 new cases of spinal cord injury reported each year. In the rat, the impact of spinal cord injury causes immediate hemorrhagic and mechanical damage to the area of injury. There is also loss of tissue in the hours and days following the initial trauma. This is better known as “secondary injury.” There are many different development parameters involved in this secondary injury, such as disruption of cellular homeostatic mechanisms, activation of intracellular messengers, as well as a variety of immune responses. Another factor of secondary injury is the over stimulation by excessive glutamate release resulting in excitotoxic cell death. Inflammation appears to be a key factor in the response to injury and probably the major cause of cell death and behavioral deficits.

The role of inflammation in spinal cord injury (SCI) is controversial and yet remains to be clearly defined. Inflammatory cells are associated with delayed neuronal death and demyelination, and they also may be integral to neural regeneration.

Current pre-clinical and clinical studies demonstrated that traumatic SCI initiates a very robust inflammatory response, both within the spinal cord and systemically. Specifically, exhibits that traumatic SCI activates NF-κB within macrophages, microglia, endothelial cells and neurons. NF-κB is a transcription factor that plays a pivotal role in regulating inflammation, and possibly apoptotic cell death pathways and induction of pro-inflammatory cytokines and chemokines, TNF-α, IL-1b and MCP-1 within the spinal cord and in activated macrophages.

The injury results in release of inflammatory mediators from neurons, microglia, astrocytes and endothelial cells. These mediators include TNF-α, IL-1, IL-6, heme oxygenase, and Cox-2. The spinal cord has limited antioxidant protection. The CNS has very limited amounts of catalase, superoxide dismutase and glutathione dismutase. There is a migration of neutrophils to the damaged area, followed by presence of numerous monocytes.

Over the next hours to days there is further destruction of neurons through apoptosis and demyelination. There is upregulation of NF-κB and growth factors (TGF-B). Glial cells proliferate and lay down a scar containing keratin sulfate interfering with regrowth of neurons past the area of damage. Mesenchymal cells induce subsequently additional scarring, which increases the lesion volume and mitigates any repair processes. Researchers have been searching for ways to alter the immune response such that the ultimate outcome of SCI will be improved. The present anti-inflammatory drug of choice and standard of care, methylprednisolone (immunosuppressant) was tried for years and was found to be ineffective in improving outcomes in SCI. No other intervention to date has been found to be effective.

Current strategies that non-selectively suppress inflammation have not improved outcomes after SCI, perhaps because chronic inflammation has increased adverse effects more than beneficial effects after SCI. A critical need exists for a more targeted immunological strategy towards chronic inflammation that should provide prolonged neuroprotection, limiting, or eliminating neuronal damage and improved neurological outcomes in the SCI patient. Based on our knowledge of the mechanism of action of TCDO and the results of the two animal studies performed so far, we believe that TCDO is an effective treatment for acute SCI and for subsequent complications.

### Stroke

In one embodiment, the present invention provides a method of treating a subject having a stroke comprising administering to the subject a therapeutically effective amount of TCDO.

A stroke, or cerebrovascular accident (CVA), is the rapid loss of brain function due to disturbance in the blood supply to the brain. This can be due to ischemia (lack of blood flow) caused by blockage (thrombosis, arterial embolism), or a hemorrhage. Inflammation plays an important role in the pathogenesis of ischemic stroke and other forms of ischemic brain injury. Experimentally and clinically, the brain responds to ischemic injury with an acute and prolonged inflammatory process, characterized by rapid activation of resident cells (mainly microglia), production of proinflammatory mediators, and infiltration of various types of inflammatory cells (including neutrophils, different subtypes of T cells, monocyte/macrophages, and other cells) into the ischemic brain tissue. Experimentally, focal cerebral ischemia induces a time-dependent recruitment and activation of inflammatory cells, including neutrophils, T cells, and monocytes/macrophages, and inhibiting the inflammatory response, decreases infarct size and improves neurological deficit in experimental stroke. Although anti-inflammatory approaches have proven successful in animal models, attempts to translate this into clinical application have been unsuccessful, likely as a result of the heterogeneity in mechanisms underlying post-ischemic brain inflammation and the uncertain time window at which inflammation could be targeted in the human disease situation. Microglial cells, the resident macrophages of the brain, are activated rapidly in response to brain injury. Most current data have shown that blood-derived macrophages are recruited into the ischemic brain tissue, most abundantly at Days 3-7 after stroke (but not significant prior to 3 days after cerebral ischemia). The major pathway for clearance of infiltrating neutrophils and their potentially cytotoxic substances from the inflammatory sites is apoptosis followed by engulfment by activated microglia/macrophages. Macrophages can resolve neutrophils and therefore, reduce neuronal injury by triggering neutrophil apoptosis, engulfing them, and thereby preventing the release of cytotoxic substances into the surrounding tissue. Induction of apoptosis and phagocytosis of apoptotic neutrophils by reactive microglia/macrophages is a critical step in the resolution of the inflammatory response and in preventing further exacerbation of the ischemic injury. There is a growing body of evidence that inflammatory cell infiltration is predominantly deleterious in the early phase after ischemic stroke.

It is clear that macrophages play an integral role after a stroke has occurred. As such, the regulation of macrophages with an agent, such as TCDO, may be therapeutically beneficial in the treatment of stroke.

### Multiple Sclerosis (MS)

In one embodiment, the present invention provides a method of treating a subject having a multiple sclerosis comprising administering to the subject a therapeutically effective amount of TCDO.

Multiple sclerosis (MS) is a chronic inflammatory disease of the central nervous system (CNS). Major neuropathological hallmarks of MS are inflammatory demyelinating lesions associated with perivascular infiltrates containing macrophages and lymphocytes. It is widely accepted that macrophages play an important role during MS pathogenesis and both detrimental and beneficial effects of macrophages have been observed during MS and experimental autoimmune encephalomyelitis (EAE), an animal model for MS. Activated macrophages secrete many products that could contribute to axonal and oligodendrocyte damage seen in MS lesions, such as matrix metalloproteinases (MMPs), NO and glutamate. The elimination of infiltrating macrophages by clodronate liposomes suppressed clinical signs of EAE. A correlation was found between location and amount of axonal damage and loss and cellular infiltrates containing macrophages. These studies suggest that macrophages play a detrimental role, but beneficial effects have been reported for macrophages as well. Phagocytosis of myelin debris is important for axonal repair/regrowth and remyelination, since myelin components are known to be growth inhibiting. Moreover, activated macrophages/microglia are found to be sources of neurotrophins and growth factors, such as brain derived neurotrophic factor (BDNF) in MS lesions and elimination of infiltrating macrophages reduced remyelination in demyelinating models.

As such treatment using an agent which regulates macrophages, such as TCDO, may be therapeutically beneficial in the treatment of MS.

### Cardiovascular Disease/Myocardial Infarction

In one embodiment, the invention provides a method of treating a subject having a myocardial infarction comprising administering to the subject a therapeutically effective amount of TCDO. Recently microglial activation has been reported in rats with myocardial infarction. These activation was specific to brain nuclei involved in cardiovascular regulation suggesting possible role of microglial activation in pathogenesis of heart failure.

A myocardial infarction (MI) occurs when a coronary artery becomes occluded, resulting in an insufficient oxygen supply to the downstream myocardium. Following myocardial infarction (MI), circulating blood monocytes respond to chemotactic factors, migrate into the infracted myocardium, and differentiate into macrophages. At the injury site, macrophages remove necrotic cardiac myocytes and apoptotic neutrophils; secrete cytokines, chemokines, and growth factors; and modulate phases of the angiogenic response. As such, the macrophage is a primary responder cell type that is involved in the regulation of post-MI wound healing at multiple levels. Macrophages influence several wound healing events, including fibroblast activation necessary for scar formation and endothelial cell activation necessary for angiogenesis.

The primary role of the macrophage in the post-MI is to facilitate wound healing through phagocytosis of necrotic cells and secretion of growth factors and angiogenic molecules. Macrophage migration is directed by signals from the injured myocardium, including signals from resident cells (myocytes) and acute inflammatory cells (neutrophils). Activated macrophages, in turn, produce many cytokines, chemokines, and proteases such as MMPs.

Following MI, the inflammatory reaction is a prerequisite for healing and scar formation. Several laboratories have recently shown that M-CSF treatment increases macrophage infiltration post-MI, resulting in improved function and accelerated infarct repair, while macrophage depletion using clodronate-containing liposomes impaired wound healing in a cryoinjury mouse model.

As such, treatment using an agent which regulates macrophages, such as TCDO, may be therapeutically beneficial in the treatment of MI.

### Viral/Bacterial/Parasitic Infections

In one embodiment, the present invention provides a method of treating a subject having an microbial infection comprising administering to the subject a therapeutically effective amount of TCDO.

In another embodiment, the present invention provides a method of treating a subject having viral infection comprising administering to the subject a therapeutically effective amount of TCDO.

In another embodiment, the present invention provides a method of treating a subject having parasitic infection comprising administering to the subject a therapeutically effective amount of TCDO.

The innate immune system is responsible to responding to microbial or viral infections. The innate immune system reacts immediately when an infectious agent is detected and is not antigen specific.

When an infectious agent is detected, neutrophils begin a signaling cascade. Clotting system peptides, complement products and cytokines are released to begin the inflammation cascade. Macrophages and other immune cells are recruited to the site where the infectious agent was detected, triggering local inflammation. Dendritic cells also participate, engulfing foreign organisms and undergoing activation. These cells will interact with the adaptive immune system by presenting antigens to T cells. Ultimately the infectious agent is destroyed through phagocytosis.

### HIV

Macrophages play an important role in the innate immune response. As an example, macrophages play a crucial role in HIV-1 infection. They are among the first cells infected by HIV-1, and have been proposed to form a reservoir of HIV-1 in infected persons. The infection of mononuclear phagocytes with HIV-1 is an important element in the development of HIV-associated dementia complex (HAD). The only brain cell type that is “productively” infected with the virus are microglial cells. It has also become clear that neurotoxic mediators released from brain microglia play an important role in the pathogenesis of HIV-1.

“HIV-1 can enter the microglial cell via CD4 receptors and chemokine co-receptors such as CCR3, CCR5, and CXCR4, with CCR5 being the most important of these. Interestingly, humans with double allelic loss of CCR-5 are virtually immune to HIV acquired via the sexual route (though can be infected by IV transmission of CCXR4 tropic viruses). IL-4 and IL-10 enhance the entry and replication of HIV-1 in microglia through the up-regulation of CD4 and CCR5 expression, respectively. The chemokines CCL5/RANTES, CCL3/MIP-1α, CCL4/MIP-1β, all of which bind to CCR5, are inhibitory to HIV-1 replication in microglial cells, apparently by their ability to block viral entry.”

Infected microglia contain viral particles intracellularly There is a correlation between the severity of dementia and microglial production of neurotoxins.

One discrepancy in HAD is the limited number of HIV-1 infected microglia in comparison to the many CNS abnormalities that occur. This suggests that chemical factors that are released from microglial cells are contributing to neuronal loss. “It has become more and more apparent that HIV-1 infected microglial cells actively secrete both endogenous neurotoxins such as TNF-α, IL-1β, CXCL8/IL-8, glutamate, quinolinic acid, platelet activating factor, eicosanoids, and NO as well as the neurotoxic viral proteins Tat, gp120, and gp41.”

Microglia are the main target of HIV-1 in the brain. When activated by HIV-1 or viral proteins, they secrete or induce other cells to secrete neurotoxic factors; this process is accompanied by neuronal dysfunction (HAD).

In an in vivo study, the effects of TCDO on cellular HIV replication machinery and the consequences of TCDO infectivity of HIV virons were evaluated. Virus yields in supernatants of TCDO-supplemented cultures of HIV-infected cells or virus infectivity in TCDO treated virus stocks were quantitated by titration assays and then calculating the 50% tissue culture infectious dose. TCDO did not affect the replication of HIV in persistently infected lymphocytic and monocytic cell lines or in peripheral blood mononuclear cells. In addition, supplementation of HIV stocks with TCDO markedly decreased the infectivity of HIV particles in a concentration dependent manner. Also, the binding of gp120 envelope glycoprotein of HIV-1 to cells is blocked by pre-incubation with TCDO. Further, the inhibition of HIV replication by the reverse transcriptase inhibitor 3′-azido-3′-deoxythymidine (zidovudine) in de novo infected cell cultures was not affected by the simultaneous addition of TCDO. However, the delayed virus spread of HIV in cultures in the presence of suboptimal concentrations of zidovudine could significantly be blocked by the simultaneous addition of TCDO. Additionally, TCDO failed to induce the chromosomally integrated HIV-1 provirus in the T-lymphoma cell line ACH2. TCDO appears to inactivate HIV particles directly, but has no influence on the intracellular replicative machinery of HIV. The results suggest that a clinical evaluation of the TCDO complex as chemotherapy for HIV infection and full-blown AIDS should be considered, particularly in patients concomitantly receiving zidovudine.

### Herpes Simplex Virus

Herpes simplex virus (HSV) can cause herpes encephalitis in babies and immunocompetent adults. Studies have shown that long-term neuroimmune activation persists after the herpes infection in patients. Microglia produce cytokines that are toxic to neurons; this may be a mechanism underlying HSV-related CNS damage. It has been found that “active microglial cells in HSV encephalitis patients do persist for more than 12 months after antiviral treatment.”

### Bacterial Infections

Lipopolysaccharide (LPS) is the major component of the outer membrane of a gram-negative bacterial cell wall. LPS has been shown to activate microglia in vitro and stimulates microglia to produce cytokines, chemokines, and prostaglandins. Although LPS has been used as a classic activating agent, a recent study of rat microglia demonstrated that prolonged LPS exposure induces a distinctly different activated state from that in microglia acutely exposed to LPS.

Streptococcus pneumonia

Streptococcus pneumonia is the most common cause of bacterial meningitis. It is primarily localized to the subarachnoid space while cytokines and chemokines are produced inside the blood brain barrier. Microglia interact with streptococcus via their TLR2 receptor; this interaction then activates microglia to produce nitric oxide which is neurotoxic. The inflammatory response, triggered by microglia, may cause intracerebral edema.

Plasmodium falciparum

Plasmodium falciparum (vector mosquito) is a parasite that causes malaria in humans. A serious complication of malaria is cerebral malaria (CM). CM occurs when red blood cells break through the blood brain barrier causing microhemorrhages, ischemia and glial cell growth. This can lead to microglial aggregates called Durck's granulomas Recent research has indicated that microglia play a major role in the pathogenesis of CM.

Other examples of parasitic infections which may be beneficially treated with TCDO include, but are not limited to, Lyme's disease non-responders/chronic, Dengue Fever, Leschmaniasis and West Nile virus.

It is clear that macrophages have an integral role in treating bacterial, viral and parasitic infections. As such, the regulation of macrophages with an agent such as TCDO could be therapeutically beneficial to a subject suffering from a treating bacterial, viral and parasitic infection.

### Wound Healing/Repair

One embodiment of the present invention provides a method of promoting wound healing/repair in a subject comprising administering to the subject a therapeutically effective amount of TCDO.

In an additional embodiment, the present invention provides a method of treating a subject having diabetes mellitus type II comprising administering to the subject a therapeutically effective amount of TCDO.

In a further embodiment, the present invention provides a method of treating a subject having ulcerations comprising administering to the subject a therapeutically effective amount of TCDO. In one aspect, the ulcerations are the result of diabetes mellitus type II.

Wound healing, or cicatrisation, is an intricate process in which the skin (or another organ-tissue) repairs itself after injury. The classic model of wound healing is divided into three or four sequential, yet overlapping, phases: (1) hemostasis (not considered a phase by some authors), (2) inflammatory, (3) proliferative and (4) remodeling. Upon injury to the skin, a set of complex biochemical events takes place in a closely orchestrated cascade to repair the damage. Within minutes post-injury, platelets (thrombocytes) aggregate at the injury site to form a fibrin clot. This clot acts to control active bleeding (hemostasis).

Macrophages are essential for wound healing. They replace PMNs as the predominant cells in the wound by two days after injury. Attracted to the wound site by growth factors released by platelets and other cells, monocytes from the bloodstream enter the area through blood vessel walls. Numbers of monocytes in the wound peak one to one and a half days after the injury occurs. Once they are in the wound site, monocytes mature into macrophages. The spleen contains half the body's monocytes in reserve ready to be deployed to injured tissue.

The macrophage's main role is to phagocytize bacteria and damaged tissue, and they also debride damaged tissue by releasing proteases. Macrophages also secrete a number of factors such as growth factors and other cytokines, especially during the third and fourth post-wounding days. These factors attract cells involved in the proliferation stage of healing to the area, although they may restrain the contraction phase. Macrophages are stimulated by the low oxygen content of their surroundings to produce factors that induce and speed angiogenesis. They also stimulate cells that reepithelialize the wound, create granulation tissue, and lay down a new extracellular matrix. By secreting these factors, macrophages contribute to pushing the wound healing process into the next phase.

Patients with diabetes mellitus (DM) type II often develop foot ulcers which are notoriously difficult to treat. The healing process may be helped by promoting anti-infectivity in wounds by enhancing phagocytosis via macrophage and presentation granulocyte activation. Better cell-mediated response for Th-1, Th-2 and also NK cells. Activated macrophages interact with fibroblasts resulting in fast formation of new granulation tissue, promotes wound healing by ‘enhancing Macrophage Activation’.

Enhanced activated macrophages express genes leading to neo-angiogenesis (tissue oxygenation). Also, neuropathy often associated in DM ulcer patients may lead to neurogenesis and myelin sheath repair. TCDO may demonstrate decrease in TNF-a via Nf-κB and cytokine pathway and proper mediation of the chronic inflammatory response prior to wound formation and ulcer. Macrophages, microglia, endothelial cells and neurons. NF-κB is a transcription factor that plays a pivotal role in regulating inflammation, and possibly apoptotic cell death pathways and induction of pro-inflammatory cytokines and chemokines, TNF-a, IL-1b and MCP-1. As such, a treatment method which regulates macrophages, such as TCDO, may be therapeutically useful in treating wounds and ultimately wound healing.

### Pain Cessation/Chronic Pain

One embodiment of the present invention provides a method of treating a patient in need of pain cessation comprising administering a therapeutically effective amount of TCDO.

Pain is an unpleasant sensory and emotional experience associated with actual or potential tissue damage, or described in terms of such damage. For some time it has been recognized that inflammatory mediators released from immune cells can contribute to these persistent pain states.

Activated macrophages —either resident or recruited from the blood by chemotactic cytokines—have been reported to contribute to experimental pain states. They can release many inflammatory mediators, notably pro-inflammatory cytokines (particularly tumor necrosis factor-α (TNFα) and interleukin-1β (IL-1β)), nerve growth factor (NGF), nitric oxide (NO) and prostanoids. When macrophages have been depleted from or recruited to peripheral tissues experimentally, they have been shown to contribute significantly to the inflammatory pain that is produced by zymosan and the irritant acetic acid. In addition, macrophage activation seems to have a marked effect on the subsequent recruitment and activation of other cell types (neutrophils) to the site of inflammation. There is an increasing awareness that anti-inflammatory pathways might be important in limiting the duration of inflammatory responses.

Damage to peripheral nerves can precipitate neuropathic pain. Such pain is often persistent and is poorly treated by existing therapies because distinct mechanisms are triggered by neuronal injury. Neuropathic pain arises as a result of many forms of nerve damage, including diabetic neuropathy, HIV neuropathy, post-herpetic neuralgia, drug-induced neuropathy and traumatic nerve injury. In damaged peripheral nerves, as in other tissues, macrophages are recruited by chemotactic molecules in the microenvironment.

It is clear that macrophages as part of the inflammatory pathway play a role in pain. As such, the regulation of macrophages, such as TCDO, may be therapeutically beneficial for pain cessation.

### Pharmaceutical Formulation

Pharmaceutical formulations of the invention may include other agents in addition to the active ingredient. For example, a suitable pharmaceutical composition for injection may comprise a buffer (e.g. acetate, phosphate or citrate buffer), a surfactant (e.g. polysorbate), optionally a stabilizer agent (e.g. human albumin), etc.

TCDO is a unique chlorite solution. TCDO has been analytically characterized as an aqueous solution containing chlorite ion (4.25%) as the active principle, and the inactive ingredients chloride (1.9%), chlorate (1.5%) and sulfate (0.7%) ions, with sodium as the cation. One milliliter of TCDO contains approximately 0.1 g of TCDO in water for injection (Ph.Eur./USP), equivalent to approximately 4.25 mg (63 μmol) of chlorite ion (ClO2-). The molarity is calculated by the chlorite ion content: TCDO contain chlorite at concentrations of 693 mM and 63 mM, respectively.

### Methods of Treatment

Therapeutic agents can be administered by parenteral, topical, intravenous, oral, subcutaneous, intraarterial, intracranial, intraperitoneal, intranasal or intramuscular means for prophylactic and/or therapeutic treatment. While all these forms of administration are clearly contemplated as being within the scope of the invention, a form for administration would be a solution for injection, in particular for intravenous or intraarterial injection or drip. In one aspect TCDO is administered by parenteral, topical, intravenous, oral, subcutaneous, intraarterial, intracranial, intraperitoneal, intranasal or intramuscular means.

TCDO is typically administered to a patient for at least one cycle. In one embodiment of the present invention, one cycle consists of administering to a patient about 5 consecutive daily doses of TCDO, then at least about 16 days of no treatment. In one aspect, a subject is administered at least one cycle of TCDO treatment. In an additional aspect a subject may be administered 1, 2, 3, 4 5, 6, 7, or more cycles as needed. One embodiment of the present invention provides that TCDO may be administered to a subject in a dose of about at least 0.1 mL/kg body weight/day to about at least 0.5 mL/kg body weight/day. In a preferred aspect TCDO is administered in a dose of about 0. 5 mL/kg body weight/day to at least 1.5 mL/kg body weight/day. In a further aspect, TCDO is administered in a dose of about at least 0.5 mL/kg body weight/day to about at least 0.75 mL/kg body weight/day. In one aspect, TCDO is administered in a dose of about at least 0.5 mL/kg body weight/day.

The following examples are intended to illustrate but not limit the invention.

### Example 1

### Vascular Events after Spinal Cord Injury: Contribution to Secondary Pathogenesis

### Methods and Materials

Animals: Female Sprague-Dawley rats (Harlan Sprague-Dawley, Indianapolis, Ind.), weighing 200-225 grams each were given free access to food and water before the experiment. The animals were anesthetized with ketamine (80 mg/kg) and xylazine (10 mg/kg). Brain temperatures were monitored using a rectal thermometer. The animals' body temperature was maintained at 37° C. by using a water jacketed heating pad. Brain temperature was monitored for 1 hour prior to injury to 6 hours following injury and was recorded at 30-minute intervals.

Experimental Groups. The spinal cord injury model utilized in the studies is described in detail (Rabchevsky et al, 2002; J. Neurosci. Res. 68:7). Young adult female Sprague-Dawley rats received a spinal cord contusion using the Precision Scientific Inc. pneumatic impactor. Female animals were used due to the paralysis associated with the injury and ease of voiding the bladder. Prior to surgery, rats were assigned to different treatment groups based on a randomized block design so that on any given surgery day all treatment groups were included. The rats were anesthetized with ketamine (80 mg/kg) and xylazine (10 mg/kg) before laminectomy was performed at the 10^{th} thoracic vertebra (T₁₀). The vertebral column was stabilized with angled clamps on the upper thoracic (T8) and lumber (T11) levels and the impactor with a tip diameter of 2 mm was delivered at approximately 50 kdynes onto the exposed, intact dura overlying the dorsal spinal cord. The impactor was immediately removed, the wound irrigated with saline and the muscle and skin openings sutured together.

Drug Administration. Compound was administered in the following paradigm: intravenous injection of saline or drug solutions 15 minutes following injury at 0.5 ml/kg (1×10⁴, 5×10⁴, and 10×10⁴ TCDO). Drug was administered once per day starting at the indicated times and continued for 5 days. The saline control group was run in parallel with the drug-treated groups.

Behavior Analysis: Basso, Beattie and Bresnahan Locomotor Rating Scale. For behavioral analysis, animals were tested prior to surgery and at 1, 2, 3, 4, 5 and 6 weeks after surgery. Animals were placed in an open field chamber (40 cm×40 cm×40 cm, 25 cm wall height) for 4 minutes to assure that all subjects obtained a maximum score of 12 using the modified Basso, Beattie, and Bresnahan (BBB) locomotor rating scale (Ferguson et al., 2004; J. Neurotrauma 21:1601; see below). Rats were placed in the open field for 4 minutes and videotaped for scoring.

Histology. At the end of the study, the animals were killed and the spinal cord fixed in 4% paraformaldehyde. For analysis, 20 μm cryosections were stained for eriochrome cyanine (EC) to differentiate between white matter and cell bodies to calculate the amount of spared tissue through the lesion site. Immunocytochemical analysis was performed on the tissue. Tissue sparing was determined by computed image analysis from 10 evenly spaced sections through the injured T10 segment. The volume of necrotic tissue divided by the total cross-sectional volume is converted to a percentage and subtracted from 100%. Sections were taken for immunocytochemical analysis for macrophage infiltration

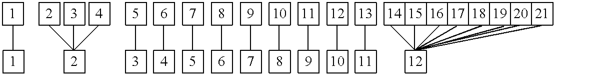

(CD68), astrocyte activation. Modified BBB scale based on Ferguson et al., 2004.

Exclusion of Animals From the Study. Animals were excluded from the study based upon several criteria: (1) Animals died prior to completion of study (at any point). Data collected to the time of death was provided. (2) Animals developed seizure-like activity following injury. (3) Excessive bleeding was detected during or immediately following injury. (4) To take in account significant variations in the severity of the initial impact injury: if an animal within a group showed a lesion volume that was 2 folds higher than the standard deviation of its particular group.

Statistical analysis. The results were expressed as the mean±standard deviation. The significance of difference in the behavioral and histological data was analyzed using unpaired t-test, two-tailed with 95% confidence intervals comparing each SCI-treated group to SCI-saline group. In this particular study, NTS usual ANOVA analysis could not be used since this was not a dose-response study and comparative analysis between all drug-groups would have been irrelevant.

Spinal Cord injury Rat Model

| | No. | Com- | Dose (units | Application | |
|---|---|---|---|---|---|
| Group | of rats | pound | TCDO) | (start time) | Dose/Volume |
| 1 | 10 | Saline | NA | IV | 0.5 ml/kg |
| 2 | 10 | IBT-1 | 1 × 10⁴ | IV (15 min) | 0.5 ml/kg |
| 3 | 10 | IBT-1 | 5 × 10⁴ | IV (15 min) | 0.5 ml/kg |
| 4 | 10 | IBT-1 | 10 × 10⁴ | IV (15 min) | 0.5 ml/kg |
| 5 | 10 | Control | NA | IV (15 min) | 0.5 ml/kg |

*Not injured.
Group 1: Injured - 0.5 ml/kg/day saline
Group 2: Injured - 0.5 ml/kg/day, TCDO
Group 3: Injured - 0.5 ml/kg/day, TCDO
Group 4: Injured - 0.5 ml/kg/day, TCDO
Group 5: Not injured - 0.5 ml/kg/day saline

Each animal received induction of 5 i.v. injections for a total of 5×0.5 ml/kg of body weight. Solutions were infused intravenous to the animals once per day for 5 days (which designates one treatment cycle). The solutions were administered by slow infusion under anesthesia. The test compound was diluted 20 mls to 250 mls of normal saline (0.09% Na Cl—) (actual dilution was 1 mls to 12.5 mls) prepared daily for infusion. Diluted compound was used and discarded.

Endpoints. (1) Behavioral deficits (2) Histological analysis (3) Effects of TCDO solutions on the protection from spinal cord injury. Animals were evaluated for spinal cord injury. All animals in the test groups were dosed as indicated above.

RESULTS. Spinal cord injury in rat. The relative severity of SCI in these studies was assessed. Data were from rats with SCI that were intravenously injected with vehicle or invention compound (TCDO, WF-10).

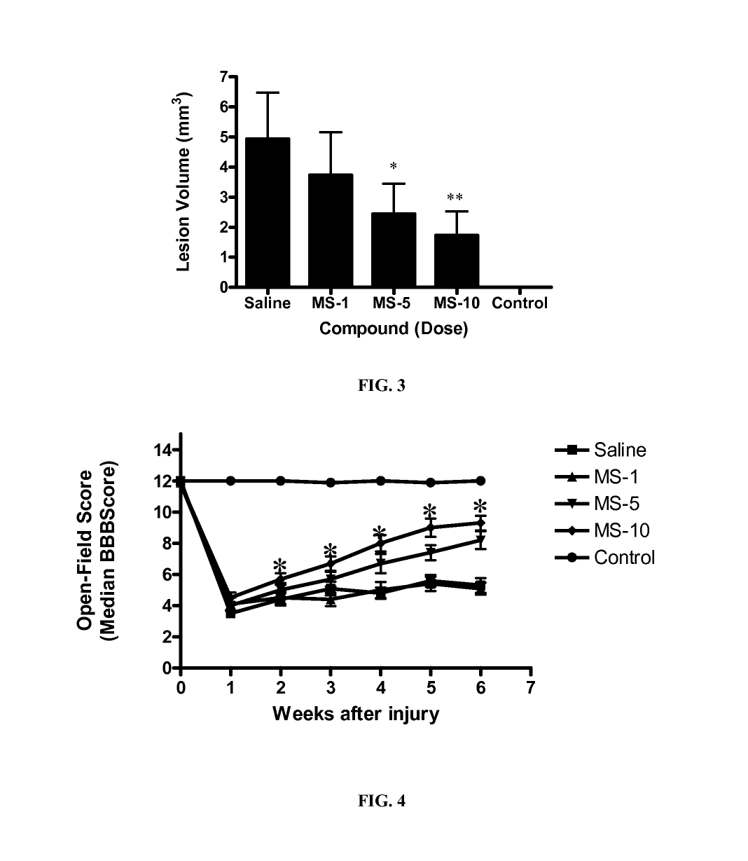

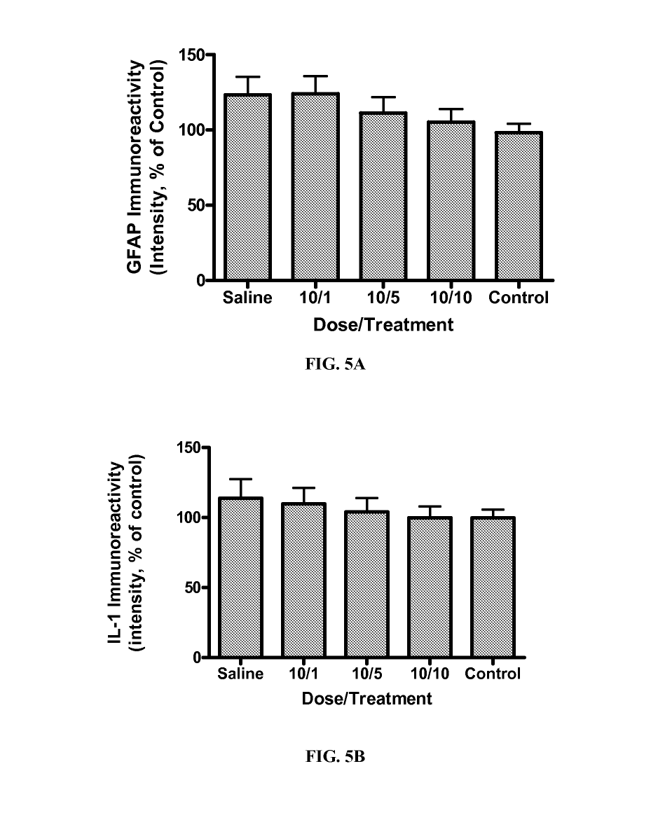

Lesion volume. As shown in Table 1 and FIG. 3, treatment with TCDO reduced the lesion volume by 25, 50 and 65% when delivered at doses of 1×10⁴, 5×10⁴, and 10×10⁴ TCDO starting at 15 minutes following injury, respectively. The changes were statistically significant relative to the Saline group (group 1) when compared to the 5×10⁴ and 10×10⁴ doses. Animals treated with the 1×10⁴ dose did not show a significant reduction of the lesion volume when compared to control animals (group 1).

TABLE 1
Percent decrease in lesion volume in the spinal cord.

| | | | | Lesion | Percent |
|---|---|---|---|---|---|
| | | | Com- | volume (mm³) | change in |
| Group | Dose | ID | pound | P value | Lesion volume |
| 1 | 0.5 ml/kg | 1 | Saline | 4.932 ± 1.538 | NA |
| 2 | 0.5 ml/kg | 2 | WF-10 | 3.726 ± 1.427 | 24.5% |
| | | | 1 × 10⁴ | 0.0858 | reduction |
| 3 | 0.5 ml/kg | 3 | WF-10 | 2.444 ± 0.9960 | 50.5% |
| | | | 5 × 10⁴ | 0.0004 | reduction |
| 4 | 0.5 ml/kg | 4 | WF-10 | 1.722 ± 0.840 | 65.1% |
| | | | 10 × 10⁴ | <0.0001 | reduction |
| 5 | 0.5 ml/kg | 5 | Saline | 0.0 ± 0.00 | 100% |
| | | | | NA | reduction |

Percent changes are compared to the Saline group (group 1)

Mortality: There were no deaths in this study.

Behavioral Measurements. Animals were assessed in an open field chamber (40 cm×40 cm×40 cm, 25 cm wall height) for 4 minutes to assure that all subjects could obtained a maximum score of 12 using the modified Basso, Beattie, and Bresnahan (BBB) locomotor rating scale. Rats were placed in the open field for 4 minutes and videotaped for scoring. As seen in FIG. 4, prior to injury none of the rats had deficits, on day 7 all animals showed approximately the same deficit in movement due to the injury. As seen in FIG. 4, treatment with WF-10 at 1×10⁴ dose treated group had no significant effect on motor recovery after SCI (compared to saline treated group). However, the 5×10⁴ and 10×10⁴ treatments significantly improved locomotor recovery on day 14 through 42, post-SCI relative to the saline treated group.

TABLE 2
Locomotor recovery (BBB score) on days 14, 28 and 42 post-injury- Raw data

| | | | Day 14 | Day 28 | Day 42 |
|---|---|---|---|---|---|
| | | Com- | Mean ± SD | Mean ± SD | Mean ± SD |
| Group | Dose | pound | P-value | P-value | P-value |
| 1 | 0.5 ml/kg | Saline | 4.400 ± 0.400 | 4.800 ± 0.359 | 5.300 ± 0.473 |
| | | | NA | NA | NA |
| 2 | 0.5 ml/kg | WF-10 | 4.500 ± 0.401 | 5.000 ± 0.516 | 5.100 ± 0.379 |
| | | (1 × 10⁴) | NS | NS | NS |
| 3 | 0.5 ml/kg | WF-10 | 5.000 ± 0.447 | 6.700 ± 0.616 | 8.200 ± 0.574 |
| | | (5 × 10⁴) | <0.05 | <0.05 | <0.05 |
| 4 | 0.5 ml/kg | WF-10 | 5.700 ± 0.367 | 8.00 ± .538 | 9.300 ± 0.473 |
| | | (10 × 10⁴) | <0.05 | <0.05 | <0.05 |
| 5 | 0.5 ml/kg | Control | 12.0 ± 0.00 | 12.0 ± 0.00 | 12.0 ± 0.00 |
| | | | <0.05 | <0.05 | <0.05 |

Inflammatory markers analysis. Following injury at the end of the experiment, animals were assessed for inflammation using immunohistochemical analysis for glial fibrillary acidic protein (GFAP, astrocytes), Interleukin-1 (IL-1, cytokines), and CD-68 (macrophage, microglial cells). When examined GFAP showed a significant decrease when WF-10 was administered at 5×10⁴ or 10×10⁴ TCDO. However, at 1×10⁴ TCDO there was no significant effect. There were no significant changes in IL-1 levels at 6 weeks following SCI except at the 5×10⁴ and 10×10⁴ dose and comparison of the saline treated group to the control (non-injured) group (saline group was higher). In addition, the CD-68 immunostaining demonstrated a significant decrease in CD-68 at the 5×10⁴ and 10×10⁴ dose groups and control group.

TABLE 3
Inflammatory Marker Analysis

| | | | GFAP | IL-1 | CD-68 |
|---|---|---|---|---|---|
| | | Com- | Mean ± SD | Mean ± SD | Mean ± SD |
| Group | Dose | pound | P-value | P-value | P-value |
| 1 | 0.5 ml/kg | Saline | 123.3 ± 11.99 | 113.8 ± 13.64 | 125.3 ± 16.41 |
| | | | NA | NA | NA |
| 2 | 0.5 ml/kg | WF-10 | 124.1 ± 11.57 | 109.8 ± 11.37 | 121.3 ± 13.45 |
| | | (1 × 10⁴) | 0.7091 | 0.2166 | 0.3072 |
| 3 | 0.5 ml/kg | WF-10 | 111.3 ± 10.61 | 104.0 ± 9.883 | 106.0 ± 8.807 |
| | | (5 × 10⁴) | 0.0001 | 0.0023 | <0.0001 |
| 4 | 0.5 ml/kg | WF-10 | 105.1 ± 8.737 | 99.94 ± 7.935 | 99.82 ± 9.326 |
| | | (10 × 10⁴) | <0.0001 | <0.0001 | <0.0001 |
| 5 | 0.5 ml/kg | Control | 98.24 ± 5.818 | 99.87 ± 5.888 | 98.65 ± 5.849 |
| | | | <0.0001 | <0.0001 | <0.0001 |

DISCUSSION. The results show that combined treatments with TCDO appears to provide protection from spinal cord injury. The studies performed here demonstrate a dose dependent effect of TCDO treatment to protect the spinal cord from the detrimental effects of SCI significantly reducing the lesion volume and significantly improving locomotor recovery at 5×10⁴ and 1×10⁵ TCDO. When administered intravenously, treatment with TCDO was found to be protective against spinal cord injury in the rat.

### Example 2

### Efficacy of TCDO in the Treatment of Type II Diabetic Ulcer Patients

A multicenter, randomized, two-arm, confirmatory Phase 3 study to evaluate the efficacy and safety of IBT's test treatment drug, an intravenous solution in late-stage Type II diabetic ulcer patients disease.

Objectives: The primary objective is to evaluate the effect of intravenous (IV) administration of IBT's test treatment drug on clinical progression in Type II diabetic ulcer patients. Patients receiving or have received combination acceptable/approved standard of care therapy. Clinical progression is defined as any occurrence of a new adult Type II diabetic ulcer patients-defining condition, recurrence of selected adult Type II diabetic ulcer patients-defining conditions, failure of wound closure, continued progression, amputation or death. The secondary objectives are to evaluate the effect of IBT's test treatment drug administration on number, duration, and cause of hospitalizations, and immunologic, neurogenic, markers and to evaluate long-term safety and tolerability of IBT's test treatment drug.

Number of Patients. This study will enroll n=100 patient-Type II diabetic ulcer patients—who are or not taking any acceptable/approved standard of care therapy for diagnosis of diabetic ulcer ranging from greater than for short term, proof of concept. After which, will be followed by a larger trial of 900-patient Type II diabetic ulcer patients Phase 3 clinical trial, randomized, placebo controlled, double-blind study, n=900 patients. Powered for significance.

Study Design. This is a confirmatory, multicenter, randomized, two-arm clinical trial to evaluate the safety and clinical effect of four treatment cycles of 0.5 mL/kg IBT's test treatment drug in the treatment of adult patients with Type II diabetic ulcer patients. After patients are screened and successfully meet the study criteria, they will be randomized to one of the two treatment groups as follows:
Standard of care therapy plus IBT's drug treatment (four treatment cycles of 0.5 mL/kg): initial first 100 patients for 2 cycles of test drug.
Naive patients with no standard of care therapy plus IBT's test treatment drug: 300 patients.
Each patient will be assigned to one of the two treatment groups using a 1:1 (standard of care: standard of care plus IBT's test treatment drug) randomization scheme.
Patients will be monitored throughout the study for clinical progression (primary efficacy endpoint) and number, duration, and cause of hospitalizations and immunological marker (secondary efficacy endpoints). Events will be considered endpoints starting at Week 5 (after the second treatment cycle with IBT's test treatment drug).
Patients randomized to the treatment arm assigned to receive standard of care alone who have reached a confirmed primary endpoint will be offered four cycles of open-label IBT's test treatment drug once their diabetic ulcer type has re-stabilized by closure of ulcer/wound type. All patients who receive IBT's test treatment drug, including those who receive it with an open-label, will be evaluated for safety during the treatment period and 13 weeks after the last infusion of IBT's test treatment drug evaluating secondary D2 associated effects/endpoints: glucose level, weight gain/loss, reduction/increase of nerve sensation, retinopathy, renal and pancreatic evaluation.
The duration of the treatment period will be 11 weeks. Patients will be evaluated at screening, then at Weeks 1, 4, 7, 10, 13, 16, 20, and 24.
The statistical power of this study is based upon frequency distributions of adult Type II diabetic ulcer patients-defining event, amputation or no effect of the clinical trial within the 24-week period.

The study will be terminated and the endpoints will be assessed when the final surviving patient to receive treatment has completed the Week 24 follow-up evaluation.

Patients will continue to receive pre-existing acceptable/approved standard of care treatment alone or in addition to IBTs test treatment drug, which will be administered by intravenous infusion for four treatment cycles of 5 consecutive days per cycle, each cycle separated by a 16-day interval. IBT's test treatment drug treatment cycles will be administered from Days 1 to 5, Days 22 to 26; Days 43 to 47; and Days 64 to 68.

Patients randomized to the treatment arm assigned to receive standard of care alone who have reached a primary endpoint, which has been confirmed, will be offered four cycles of IBT's test treatment drug in an open-label.

Criteria for Evaluation: This study will evaluate the effects of IBT's test treatment drug on clinical progression, defined as any occurrence of a new adult Type II diabetic ulcer patients-defining condition, recurrence of selected adult Type II diabetic ulcer patients-defining conditions, active progression of wound and clinical conditions determined by Safety Committee o case by case per Medical Monitor and attending physician and staff, death and number, duration, and cause of hospitalizations, and immunological markers (will be determined) (secondary efficacy endpoints). The safety and tolerability of IBT's test treatment drug will be evaluated by analysis of adverse events and standard clinical laboratory tests.

### Example 3

### Use of TCDO for the Treatment of SCI

Tetrachlorodecaoxygen (TCDO) was tested for efficacy in the spinal cord injury (SCI) rat model. In order to determine the efficacy of TCDO, rats were subjected to spinal cord injury and 6 weeks of recovery. Rats were injected intravenously with saline or TCDO solutions. Rats received 1 injection regiment: administered 15 minutes after injury. Animals were injected once daily for 5 days following the injury. Treated rats were examined after 1, 2, 3, 4, 5, and 6 weeks for drug behavioral effect. The animals were examined for changes in lesion volume (LV) and Basso, Beattie and Bresnahan (BBB) locomotor test. Administration of TCDO at 15 minutes following injury showed a dose dependent decrease in the LV and improvement in the BBB score. Administration of TCDO initiated at 15 minutes following injury decreased the LV by 49% and improved the BBB score by 158%. However, by 7 days, TCDO was not effective in reducing the LV or behavioral deficits. These data suggest that TCDO treatment is protective in the spinal cord by i.v. injection following injury in the rat model of spinal cord injury (SCI).

### Example 4

### Use of TCDO for Treatment of Non-Healing Wounds

TCDO was investigated for its effects on non-healing wounds. In the study, 271 patients with non-healing wounds which included post-operative wounds, venous ulcers, arterial ulcers, post traumatic wounds and decubitus ulcers. The patients received either 5 mL of TCDO or saline twice daily applied by compress or directly to the wound for 21 days. TCDO was significantly superior to saline for wound cleaning and formation of new tissue. Irrespective of wound types, wound surface decreased 2 to 4 times more quickly with TCDO treatment compared to saline. The best results were observed in chronic ulcers and post operative wounds. Adverse events were reported by 7% and 12% of patients treated with saline and TCDO respectively, the most frequent was slight pain.

### Example 5

### Determining the Clinical Dose for TCDO

HIV-positive patients received one cycle of intravenous infusions of WF10—defined as 5 consecutive daily doses followed by 16 days of no treatment—at a dose of 0.5L/kg body weight/day to 50 μL, (0.5 mL)/kg body weight/day, without apparent adverse effect. Patients were then given 4 cycles of WF10 with the daily dose ranging from 0.5 mL/kg body weight to 1.5 mL/kg body weight. The maximal tolerated dose of WF10 was determined to lie between 0.5 mL/kg body weight to 0.75 mL/kg body weight per day. Doses of 0.75 mL/kg body weight and greater were associated with phlebitis and a pattern of decreased hemoglobin. However, in this and subsequent clinical studies, administration of 4 cycles of 0.5 mL TCDO/kg body weight per day showed no clinically relevant hemolysis.

### Example 6

### Use of TCDO for Diabetic Foot Ulcer Therapy

This randomized controlled trial was undertaken to evaluate the effect of TCDO as an adjunct to the standard treatment of diabetic foot ulcer. A total of 40 participants were randomized into 2 groups of 20. One group underwent standard therapy combined with infusions of TCDO, and 1 underwent standard therapy combined with placebo. The wound severity scores, which vary with the severity of infection and inflammation, necrotic and granulation tissues, and wound depth and area, were assessed weekly for 9 weeks. Before treatment, the wound severity scores were not significantly different statistically between the 2 groups (13.7±2.8 and 12.9±3.2). After 9 weeks, the TCDO group had a statistically significant decreased wound severity score compared with that of the placebo group (1.8±1.9 versus 4.4±5.3, respectively, p<0.05). Subgroup analyses comparing the TCDO and placebo groups showed statistically significant decreases of infection and inflammation (0.0±0.0 versus 0.8±0.9, respectively, p<0.01), necrotic tissue (0.0±0.0 versus 0.8±1.1, respectively, p<0.01), and an increase of the amount of granulation tissue (0.1±0.3 versus 0.8±1.2, respectively, p<0.05). The wound depth and wound area also decreased more in the TCDO group; however, these decreases were not statistically significant. No severe adverse events were observed throughout the observation period. We concluded that the addition of TCDO to standard wound care statistically significantly reduced the wound severity score, infection and inflammation, and necrotic tissue and enhanced the formation of granulation tissue.

While preferred embodiments of the invention have been described, the invention is not limited to these embodiments, and the scope of the invention is defined by way of the appended claims.

What is claimed is:

1. A method of reducing and/or inhibiting proinflammatory mediator response in a subject comprising administering to the subject a therapeutically effective amount of Tetrachlorodecaoxygen (TCDO).

2. A method of modifying cellular immunity in a subject comprising administering to the subject a therapeutically effective amount of TCDO.

3. A method of controlling chronic inflammation in a subject comprising administering to the subject a therapeutically effective amount of TCDO.

4. A method of regulating macrophage function in a subject comprising administering to the subject a therapeutically effective amount of TCDO.

5. The method of any of claims 1-4 where in the subject has a macrophage mediated disease.

6. The method of claim 5, wherein the macrophage mediated disease is selected from the group consisting of spinal cord injury, traumatic brain injury, multiple sclerosis, myocardial infarction, stroke, viral infection, microbial infection, parasitic infection, chronic pain, diabetes mellitus type II, Alzheimer's disease and Parkinson's disease.

7. The method of any of claims 1-6, wherein the patient is in need of wound healing or pain cessation.

8. The method of claim 7, wherein the patient has diabetes mellitus type II.

9. A method of effecting pain cessation in a subject comprising administering to the subject a therapeutically effective amount of TCDO.

10. A method of producing an analgesic effect in a subject comprising administering to the subject a therapeutically effective amount of TCDO.

11. A method of promoting wound healing in a subject comprising administering to the subject a therapeutically effective amount of TCDO.

12. The method of any of claims 1-11, wherein TCDO is administered by parenteral, topical, intravenous, oral, subcutaneous, intraarterial, intracranial, intraperitoneal, intranasal or intramuscular means.

13. The method of any of claims 1-12, wherein TCDO is administered at a dose from about 0.1 mL/kg body weight to 1.5 mL/kg body weight.

14. The method of claim 13, wherein TCDO is administered at a dose of about 0.5 mL/kg body weight to 0.75 mL/kg body weight.

15. The method of claim 13, wherein TCDO is administered at a dose of about 0.5 mL/kg body weight.

16. The method of any of claims 1-15, wherein the subject receives at least one cycle of TCDO, wherein a cycle comprises administration of TCDO for about five consecutive days and then is not administered TCDO for at least about 16 consecutive days.

17. The method of claim 16, wherein the subject is administered 1, 2, 3, 4, 5, or 6 cycles of TCDO.
